(19) United States
(12) Patent Application Publication
(10) Pub. No.: US 2010/0086503 A1
(43) Pub. Date: Apr. 8, 2010

(54) ProDissolve

(76) Inventor: RoseMary Martin, Littleton, NC (US)

Correspondence Address: RoseMary Martin, 129 Raintree Drive, Littleton, NC, 27850, US

(21) Appl. No.: 12/287,168

(22) Filed: Oct. 7, 2008

(51) Int. Cl. A61K 8/18; A61Q 3/00

(52) U.S. Cl. 424/61

(57) ABSTRACT

The solution formula is for nail enhancements in the beauty industry. The formula is formulated to remove nail enhancements such as acrylics, gel and plastic artificial nails in a timely and safer manner for both the professional and individual. Within the formula of ProDissolve, is a fast acting natural product; which inhibits the formula to work quickly for the professional and individual.

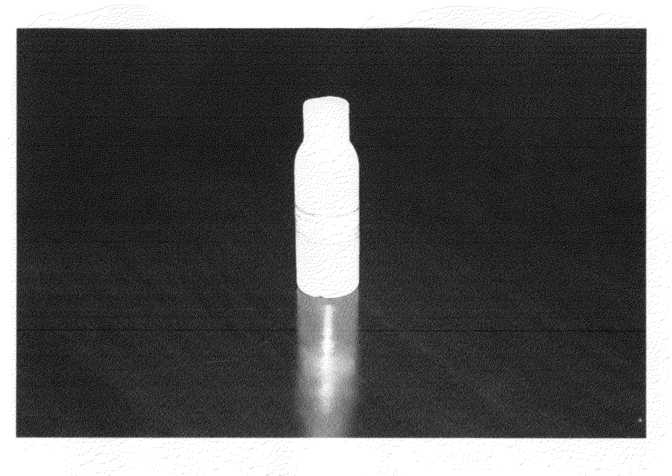

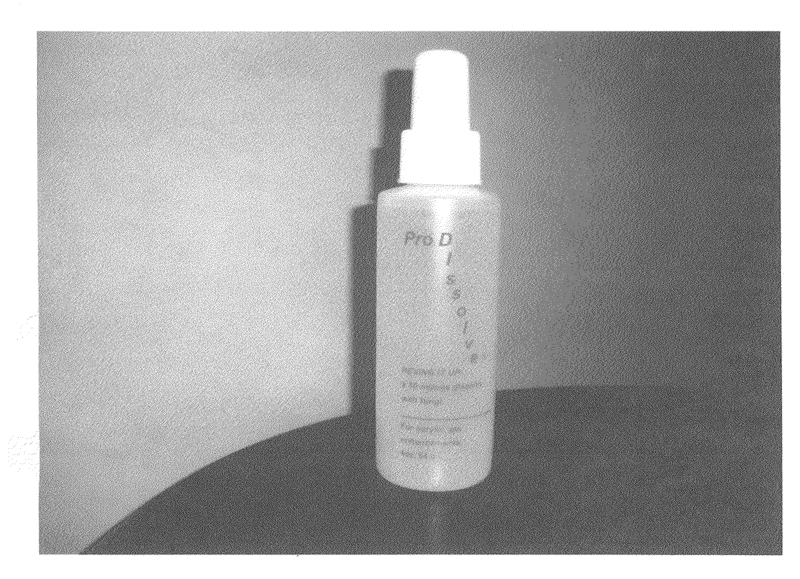

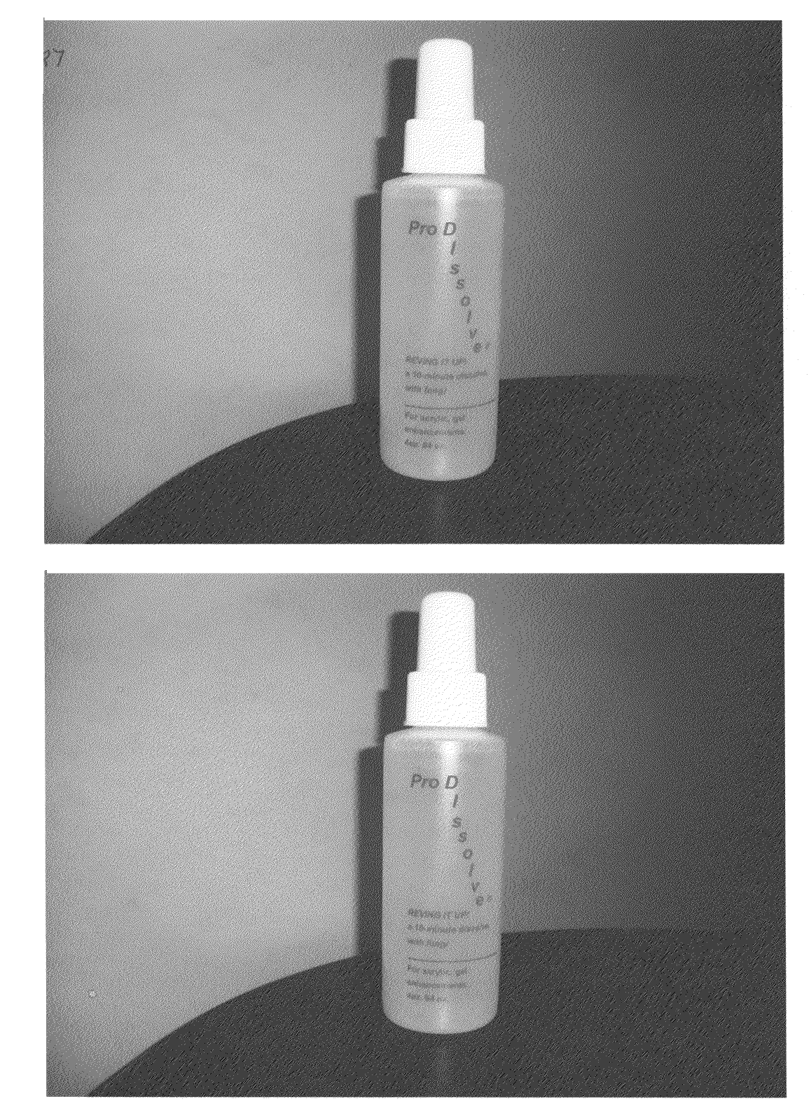

ProDissolve is an enhanced acetone-based solution for use in the beauty industry. The solution ProDissolve was found by a licensed professional while use of acrylics and during the performance of acrylic application hardening in seconds. By use of the proposed solution, ProDissolve defuses fumes in the professional and individual environment, with less fume inhalation. This product fully complies with the federal Material Safety Data Sheet (MSDS) requirements, as well as Resource Pre-manufacturing Notice (PMN) regulations. ProDissolve is more environmentally-friendly than the current wide use of pure Acetone in the beauty industry. Exposure limit according to NIOSH is 250 ppm over an eight-hour period, which is vastly more than the 10 minutes used by ProDissolve solution. ProDissolve is a product and not a waste.

### SPECIFICATION

| | | |
|---|---|---|
| 1. | ACETONE | 32 ounces |
| 2. | BAKING POWDER (enhancer) | 4½ teaspoons |
| 3. | TEA TREE OIL (fungal) | 1 drop (less than ⅛ teaspoon) |

### PROCESS

4¼ TSP. of Baking Powder mixed into 32 oz. Pure Acetone and 1 drop Tea Tree Oil (less than ⅛ tsp.) in metallic bowl. Solution is filtered then remaining ¼ tsp. Baking Powder is added and solution is then mixed.

1. The solvent solution of acetone mixed with baking powder, Tea Tree Oil, (a natural fungle ingredient), enhances to dissolve all nail enhancements in the least time, to perform a safer skill, environment and healthier solution for the nail and beauty industry.

2. The combination of solvent, mixed with baking powder, activates the solution to process; then used in a ceramic bowl to dissolve acrylic, gel and other nail enhancements in 10 minutes which is a faster rate than pure acetone solution process for dissolving enhancements.

3. ProDissolve formula is formulated for Enhancements to alleviate fungus through enhancements, as a no waste product.

4. ProDissolve is formulated to assist in fungi present, and assists in preventing present fungi.

5-9. (canceled)
